(19) United States
(12) Patent Application Publication
(10) Pub. No.: US 2010/0249485 A1
(43) Pub. Date: Sep. 30, 2010

(54) Removal of ultra-fine particles from a Fischer Tropsch Stream

(76) Inventors: Masikana Millan Mdleleni, Plattekloof (ZA); Cyril David Knottenbelt, Mossel Bay (ZA); Kgolole David Maripane, Parklands (ZA); Nkululeko Stewart Hlohloza, George (ZA)

Correspondence Address: Goodwin Procter LLP;Attn: Patent Administrator, 135 Commonwealth Drive, Menlo Park, CA, 94025-1105, US

(21) Appl. No.: 12/664,738

(22) PCT Filed: Jun. 18, 2008
(86) PCT No.: PCT/IB08/52398

§ 371 Date: May 27, 2010

Related U.S. Application Data
(60) Provisional application No. 60/929,229, filed on Jun. 18, 2007.

(51) Int. Cl. C07C 7/10

(52) U.S. Cl. 585/858; 585/833; 585/856; 585/866; 585/868

(57) ABSTRACT

This invention relates to processes for removing particles such as catalyst fines from hydrocarbon streams, such as a wax dried from a Fischer Tropsch reaction using centrifugation in combination with a treatment with an aqueous solution preferably containing an acid, or with an acid. According to an aspect of the invention, catalyst particles are removed from a wax derived from a Fischer Tropsch by pre-treating the hydrocarbon stream with an aqueous solution and forming a mixture comprising the hydrocarbon stream and 5-25% v/v organic acid solution; and introducing the mixture to a centrifuge and separating, from the mixture, a hydrocarbon stream, an aqueous solution and particles. The process may be a continuous and/or a batch process.

### BACKGROUND OF THE INVENTION

Fischer Tropsch (FT) synthesis involves the conversion of carbon monoxide and hydrogen to hydrocarbons. In the case of Low Temperature Fischer Tropsch (LTFT) synthesis, wax is the penultimate product; wax is converted by hydrocracking into shorter chains for use as high quality transportation fuels, mainly diesel fuel.

In the case of Low Temperature Fischer Tropsch (LTFT) processes the reactor is typically a Slurry Bubble Column Reactor (SBCR). Synthesis gas, a mixture of carbon monoxide and hydrogen is bubbled through a column of liquid wherein catalyst particles are suspended in the SBCR. The catalyst suspended in the liquid column catalyses the conversion of the synthesis gas to form predominantly liquid hydrocarbons. The liquid hydrocarbons (higher hydrocarbons or wax product) are removed from the SBCR by a liquid-solid separation means, normally filtration. Filters can be placed within the SBCR or externally. The catalyst particle size and filter mesh size are normally carefully selected within a specific range to compliment each other to ensure that the catalyst is retained in the SBCR or can be circulated back to the SBCR in the case of externally placed filters, further that the liquid product does not contain excessive catalyst.

Due to the extreme hydrodynamic forces within the SBCR the catalyst particles tend to undergo attrition. Attrition increases the number of fine particles (<25 microns) and reduces the average particle size. The presence of catalyst fines leads to separation difficulties can prematurely block primary filters and result in catalyst particles/fines breakthrough of the filters and become entrained in the liquid flow. Further hydroprocessing of such particle containing higher hydrocarbons (liquid wax product) will result in premature deactivation, fowling and eventual blockage of such hydroprocessing catalysts.

As per the FT catalyst art, FT catalysts are typically supported on various refractory supports such as alumina, silica and titania. Group VIII refractory supported metals are used to catalyse the FT reaction, these include cobalt, iron and ruthenium. Promoters may be added to the catalyst and could include ruthenium, palladium or platinum, rhenium, lanthanum and zirconium.

Although hydrocracking is a well-established and widely practiced technology, the prior art relating to the clean up and removal of particulate from hydroprocessing feeds is all based on crude oil—derived feeds and does not cater for FT derived feeds. FT derived feeds differ vastly from crude based feeds in that they essentially comprise of linear, paraffinic hydrocarbons, are free from sulphur, nitrogen, however, may contain traces of catalyst fines including cobalt and aluminium (alumina).

Prior art methods involve the filtering of feeds through various types of filter media. Particles down to about 1 micron can be removed, however, using large filter surfaces and with frequent replacement of filter media. This is undesirable for continuous processing.

Catalyst fines generated by various attrition mechanisms penetrate primary filter media and if not removed will contaminate the wax rundown and hinder wax upgrading processes.

Prior art technologies have been found to be unsuitable for the removal of catalyst ultra fines and portions of soluble catalyst metals.

### SUMMARY OF THE INVENTION

According to a first aspect of the invention there is provided a process for the removal of particles such as metal-containing contaminants, typically catalyst fines, from a hydrocarbon stream such as a wax derived from a Fischer Tropsch reaction, the process including the steps of
1) pre-treating the hydrocarbon stream with an aqueous solution and forming a mixture comprising the hydrocarbon stream and typically 5-25% v/v, preferably 8-12% v/v, aqueous solution; and
2) introducing the mixture to a centrifuge and separating by centrifugation, from the mixture, a hydrocarbon stream, an aqueous solution and particles.

The process may be a continuous and/or a batch process.

The aqueous solution preferably includes an acid and/or reducing agent.

The acid may be an inorganic acid such as phosphoric, hydrochloric, sulphuric, acetic, benzene sulphonic, chloroacetic acid etc.

Preferably, the acid is an organic acid, typically a carboxylic acid, preferably a monocarboxylic acid which may be branched or linear.

The organic acid preferably has a density which, in solution, is higher than the density of the hydrocarbon stream.

The organic acid may be a carboxylic acid with the following formula:

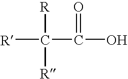

where R, R′ and R″ are each selected from H, or an aryl or alkyl group (linear, branched or cyclic) with a carbon-length of up to C₂₀.

Typically, the carboxylic acid has a total chain length of C₂-C₁₅.

Examples of organic acids are formic acid, acetic acid, propionic acid, butyric acid, oxalic acid, 2-ethyl hexanoic acid or citric acid, most preferably citric acid or 2-ethyl hexanoic acid.

Concentrations from 0.5-50% (w/w) organic acid in the aqueous solution are preferred, and concentrations of 2.5-25% (w/w) are most preferred.

The centrifuge may be operated at a pressure and temperature below the boiling point of the aqueous solution. In the case where the centrifuge is operated at atmospheric pressure, it may be operated at temperature of 60° C.—less than 100° C., typically about 98° C.

Preferably, the aqueous solution has a density which is within 250, preferably 100 kg/m³ of the density of the hydrocarbon stream. The aqueous solution may have a density of 650-850 kg/m³, preferably 700-800 kg/m³, at 98° C.

The centrifuge is preferably a continuous disc stack centrifuge or any centrifuge capable of continuous processing.

The aqueous solution from step 2) is preferably recycled to step 1).

According to a second aspect of the invention there is provided a process for the removal of particles such as metal-containing contaminants, typically catalyst fines, from a hydrocarbon stream such as a wax derived from a Fischer Tropsch reaction, the process including the steps of:
1) adding an acid to the hydrocarbon stream to form a mixture of hydrocarbon stream and acid;
2) introducing the mixture containing the hydrocarbon stream and the acid to a centrifuge and separating, from the mixture, a hydrocarbon stream, possibly an aqueous solution, and particles.

Preferably, the hydrocarbon stream separated from the mixture is washed with water.

The acid may be an inorganic acid such as phosphoric, hydrochloric, sulphuric, acetic, benzene sulphonic, chloroacetic acid etc.

Preferably, the acid is an organic acid, typically a carboxylic acid, preferably a monocarboxylic acid which may be branched or linear.

The organic acid preferably has a density which, in solution, is higher than the density of the hydrocarbon stream.

The organic acid may be a carboxylic acid with the following formula:

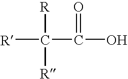

where R, R′ and R″ are each selected from H, or an aryl or alkyl group (linear, branched or cyclic) with a carbon-length of up to C₂₀.

Typically, the carboxylic acid has a total chain length of C₂-C₁₅.

Examples of organic acids are formic acid, acetic acid, propionic acid, butyric acid, oxalic acid, 2-ethyl hexanoic acid or citric acid, most preferably citric acid or 2-ethyl hexanoic acid.

The organic acid may have a concentration/purity of from 0.5-100% (w/w), preferably from 50-100% (w/), most preferably from 90-100% (w/w), typically 99.95%.

The centrifuge may be operated at a pressure and temperature below the boiling point of the aqueous solution. In the case where the centrifuge is operated at atmospheric pressure, it may be operated at temperature of 60° C.—less than 100° C., typically about 98° C.

The centrifuge is preferably a continuous disc stack centrifuge or any centrifuge capable of continuous processing.

### DESCRIPTION OF PREFERRED EMBODIMENTS

During primary filtration of Fischer Tropsch (FT) higher hydrocarbon (wax) product from the Slurry Bubble Column Reactor (SBCR), catalyst fines generated by attrition can break through the filter medium and become entrained in the wax product. This would hinder hydroprocessing, hydrocracking, hydroisomerisation, catalytic processes and even fowl distillation columns.

In broad terms this invention relates to processes for removing particles such as catalyst fines from hydrocarbon streams using centrifugation in combination with treatment with an aqueous solution preferably containing an acid, or with an acid.

Centrifugation of FT derived wax results in a reduction in ash content (total catalyst fines) from about 0.7% mass percent to about 0.2% mass percent.

According to a first aspect of the invention there is provided a process for reducing the presence of FT catalyst derived fines, ultra-fines and soluble content of a hydrocarbon stream such as a FT derived wax product by pre-treating the hydrocarbon stream with an aqueous solution and forming a mixture comprising the hydrocarbon stream and 5-25% v/v, preferably 8-12% v/v, typically 10% v/v, aqueous solution; followed by centrifugation.

According to a first preferred embodiment of the first aspect invention, the pre-treatment involves contacting a 50% citric acid solution v/v with a FT derived liquid wax at a volumetric ratio of wax to water of 1:10, followed by centrifugation in a disc stack type centrifugation system. This process results in the reduction of ash content of the wax ash content from about 0.7 mass % to about 0.003 mass % (30 parts per million (ppm) mass). The process also results in the reduction of cobalt levels in the wax from above 200 mg/kg to 5 mg/kg wax.

Process conditions for a disc stack type centrifugation system include operating at a temperature and pressure ensuring that the citric acid aqueous solution does not reach its boiling point temperature. The process may be carried out at atmospheric pressure, in which case the preferred temperature is 98° C. A 50% citric acid solution was selected since it has a density of 764 kg/m³ at 98° C. and at this density ensures good separation from the wax that has a density of about 755 kg/m³. Furthermore, citric acid has a higher density in solution than that of the wax and this enables a good separation between the wax and aqueous/acid phase (the density for citric Acid vary between 1.0 and 1.5 kg/l whereas the density of the wax is in the range of 0.6 and 0.8 kg/l at 98° C.—this assists in the separability of the 2 phases). The catalyst fines density of 1900 kg/m3 would be greater than the densities of the wax or citric acid solution. The Particle size distribution within such a wax can vary from less than 80 microns depending on the type of filter and mesh size thereof used for primary filtration.

According to a second preferred embodiment of the first aspect of the invention, the pre-treatment involved contacting 100%, 50% and 25% 2-Ethyl Hexanoic acid solutions with the liquid wax at a volumetric ratio of wax to water of 1:10, followed by centrifugation in a disc stack centrifugation system, the ash content of the centrifuged wax further reduced the wax ash content from about 0.7 mass % to less than 0.005 mass % (5 parts per million (ppm) mass). The density for the 2-Ethyl Hexanoic acid dilutions varies between 0.9 and 1 kg/l whereas the density of the wax is in the range of 0.6 and 0.8 kg/l at 98° C. This assists in the separability of the 2 phases.

Process conditions for a disc stack centrifugation system include operating at a temperature and pressure ensuring that the aqueous solution does not reach its boiling point temperature. The process may be carried out at atmospheric pressure, in which case the preferred temperature is 98° C.

According to a second aspect of the invention the presence of FT catalyst derived fines, ultra-fines and soluble content of a FT derived wax product are reduced by adding an acid to the hydrocarbon stream to form a mixture of hydrocarbon stream and acid and introducing the mixture containing the hydrocarbon stream and the acid to a centrifuge and separating, from the mixture, a hydrocarbon stream, possibly an aqueous solution, and particles. In a preferred embodiment of the invention, granular citric acid with a concentration/purity of 99.95% is added to a FT derived wax. The resulting mixture of wax containing the acid is introduced to a centrifuge and separating, from the mixture, a wax and particles. If there is water in the wax or if the citric acid is added in an aqueous solution, say at a concentration of 50-90% (w/w), an aqueous solution is also separated. Thereafter the wax is washed with water. Cobalt levels in the wax were reduced from above 200 mg/kg wax to 3 mg/kg wax. Such wax can be readily treated in downstream units without catalyst deactivation

Parameters influencing separation performance are based on a density difference between the various components present in a suspension or an emulsion. Stokes' law describes the settling velocity of a particle or droplet in a gravitational field, which in turn determines the separation efficiency.

Stokes' expression states that the separation of liquid or particles in a gravity field is not only a function of the density difference, but also of the droplet or particle size and the viscosity of the suspension. A large density difference, a large droplet size, a high gravitational force and a low viscosity all have a positive effect on separation efficiency.

The basic principle of centrifugation is to induce a high gravitational force (g-force) by rotation of the liquid, thus creating acceleration by rotation. A centrifuge generates a g-force of thousands of g, which allows rapid separation of small particles.

Such processing is performed on a continuous basis and could allow for the aqueous acidic phase to be recycled. Off line recovery of cobalt and other metals from the acidic slurry is possible by altering the pH to more alkaline conditions.

The aqueous solution may be cycled in a closed loop whereas the catalyst fines product is collected for catalyst recovery and the FT higher hydrocarbon product is allowed to rundown to tankage or direct to downstream processing units.

The invention will now be described in more detail with reference to the following non-limiting examples:

### Example 1

Wax was centrifuged with water at a water to wax ratio of 1:10, and without water to determine the effect of water alone. The following ash results as measured by ASTM D482 were obtained:

| | |
|---|---|
| Base Sample | 0.145% mass Ash |
| Centrifuged Wax | 0.108% mass Ash |
| Centrifuged Wax plus water | 0.057% mass Ash |

It should be noted that while centrifugation in water gave reduced levels of ash (contaminant) it is evident from the below examples that the addition of acid enhances the efficiency of this process.

### Example 2

### Laboratory Trials were Performed to Simulate a Disk Stack Centrifuge

Wax was filtered from a SBCR by means of a series of internally placed Pall Rigimesh™ filters.

A laboratory trial using a Petuflo™ Hotspin Centrifuge was performed to simulate an industrial scale continuous disc stack centrifuge. The advantages of the laboratory trial as opposed to the full-scale industrial trial were as follows:
Quick evaluation of samples with excellent repeatability.
The possibility to perform accurate separation tests at predetermined temperatures in order to evaluate the influence of temperature on the separation result.
An accurate method of determining the composition of a given sample, showing the content of the different insoluble fractions in percent by volume.
A means to separate the different fractions in a sample from each other in order to collect them individually for further analysis. Typical measurements are water-in-oil and oil-in water content after separation, salinity, contents of ash, sediment and particles etc.
A basis for direct scaling to a full centrifugal separator or to optimise the performance of an existing process.

### Centrifugation Trials Consisted of the Following:

Compositional spin testing in Hotspin centrifuge (60 min at 3000 rpm and 98° C.).
Low-speed spin test simulations in Hotspin centrifuge (2000 rpm and 98° C.) spin times: 2, 4, 8, 16 and 32 min.
High-speed spin test simulations in Hotspin centrifuge (3000 rpm and 98° C.) spin times: 8, 16, 32 and 64 min.

### Citric Acid Pre-Treatment:

Citric acid monohydrate (>99.5%) [C6H8O7.H2O/HOOCCH2-C(OH)(COOH)—CH2COOH.H2O] was dissolved in water (50% w/w). This aqueous solution was then added to the molten wax sample (90° C.) at a 1:10 volumetric ratio and mixed thoroughly.

### Ash Content Determination:

Ash content was determined according to ASTM D 482.

### Elemental Analysis:

Elemental content of residue after ashing was determined by acid digestion of the residue or part thereof and then followed with dilution of the digested sample with deionised water and analysed by Atomic Absorption Spectrometry.

### Spin Test Simulation:

A ‘generic’ spin test was done by running the Hotspin at 2000 rpm (98° C.) for 2, 4, 8, 16 and 32 minutes, in successive trial runs. The colour and appearance of the top phase was inspected visually. In addition, the top 5 ml from each sample tube were extracted, homogenized and analysed with respect to residual ash content.

The investigation included varying spin times, as well as mixing the wax samples with citric acid in aqueous solution. The latter greatly improved the separation result. The results are shown in Table 1.

The results from ash content analyses indicate relatively less removal of fines (ash). Ash content was reduced from about 0.7 to less than 0.3 mass % as tested by ASTM D482. However with the addition of citric acid the fines were removed more effectively reducing the ash content to as low as 30 ppm mass.

TABLE 1
Results from Hotspin centrifuging at 98°
C. (0.02 sample tubes, 5 ml of sample analysed).

| | | | | |
|---|---|---|---|---|
| | Spin | Load | Ash in | |
| Rotation | time | factor | top ph. | App of top phase |
| (rpm) | (min) | (L/h, m2) | (Wt. %) | (In liquid state) |

Low-speed spin test (no citric acid)

| | | | | |
|---|---|---|---|---|
| 2000 | 2 | 1.896 | 0.200 | Dark greyish, semi-clear |
| 2000 | 4 | 0.897 | 0.200 | Dark greyish, semi-clear |
| 2000 | 8 | 0.443 | 0.190 | Dark greyish yellow, lear |
| 2000 | 16 | 0.222 | 0.180 | Greyish yellow, clear |
| 2000 | 32 | 0.111 | 0.170 | Greyish yellow, clear |

Hiqh-speed spin test (no citric acid)

| | | | | |
|---|---|---|---|---|
| 3000 | 8 | 0.204 | 0.190 | Greyish yellow, clear |
| 3000 | 16 | 0.100 | 0.190 | Greyish yellow, clear |
| 3000 | 32 | 0.050 | 0.180 | Greyish yellow, clear |
| 3000 | 64 | 0.025 | 0.170 | Greyish yellow, clear |

High-speed spin test (with addition of citric acid)

| | | | | |
|---|---|---|---|---|
| 3000 | 8 | 0.204 | 0.020 | Light yellow, clear |
| 3000 | 16 | 0.100 | 0.009 | Light yellow, clear |
| 3000 | 32 | 0.050 | 0.037 | Light yellow, clear |
| 3000 | 64 | 0.025 | 0.003 | Light yellow, clear |

### Example 3

### Laboratory Trials Using a Petuflo™ Hotspin Centrifuge PRL050049

A laboratory trial using a Petuflo Hotspin Centrifuge was performed.

A 50% m/m aqueous citric acid solution was added to a molten wax sample at a 1:10 volumetric ratio and mixed thoroughly. Molten wax was transferred into graduated 15 ml glass centrifuge tubes. Centrifugation was performed at speeds of 3000 rpm, 3500 rpm and 4000 rpm while maintaining the internal temperature of the centrifuge at 98° C. for 15 minutes.

After centrifugation the tubes were left to cool and compared to untreated wax that had been placed in similar tubes.

Once cooled the wax pellets/tubes were removed from the centrifuge tubes and compared against each other by visual inspection (in terms of colour). Untreated wax was light gray in colour whereas the treated wax was white in colour. Cobalt levels in the wax were reduced from above 200 mg/kg wax to 5 mg/kg wax.

### Example 4

### Laboratory Trials Using a Petuflo™ Hotspin Centrifuge PRL050049

A laboratory trial using a Petuflo Hotspin Centrifuge was performed.

Molten wax was mixed with granular citric acid (99.95% w/w) and decanted into graduated 15 ml glass centrifuge tubes. Centrifugation was performed at speeds of 3000 rpm, 3500 rpm and 4000 rpm while maintaining the internal temperature of the centrifuge at 98° C. for 15 minutes. The wax was washed with copious amounts of water and left to solidify in the centrifuge tube.

The solidified wax cylinders/tubes were removed from the centrifuge tubes and compared against each other in terms of colour, and levels of residual ash or cobalt. Untreated wax was light grey in colour whereas the treated wax was white in colour.

Cobalt levels in the wax were reduced from above 200 mg/kg wax to 3 mg/kg wax. Such wax can be readily treated in downstream units without catalyst deactivation.

### Example 5

### Laboratory Trials Using a Petuflo Hotspin Centrifuge and 2-ethyl hexanoic Acid

A laboratory trial using a Heated Centrifuge was performed.

Molten wax was mixed with portions of 2-ethyl hexanoic acid at a volumetric ratio of hexanoic acid to wax of 1:10. The 2-ethyl hexanoic Acid was diluted in water in the following percentages, 100% 2-EHA, 50% 2-EHA and 25%2-ERA. Wax containing 10% portions of the various concentrations of 2-ethyl hexanoic Acid were thoroughly mixed and decanted into 100 ml glass centrifuge tubes. Centrifugation was performed at speeds of 3000 rpm, while maintaining the internal temperature of the centrifuge at 98° C. for 15 minutes.

After centrifugation the tubes were left to cool and tested for ash content as per the ASTM D482.

The Ash content of the untreated sample was 0.145% mass, centrifugation of this sample without any pre-treatment resulted in a reduction to 0.107% mass. Treatment however with a 25% solution 2-ethyl hexanoic acid in water reduced the ash content down to 0.00425% mass.

TABLE 2
2 Ethyl Hexanoic Acid

| Sample Ash (%): | |
|---|---|
| Base Wax | 0.145 |
| Centrifuged Wax | 0.107 |
| Centrifuged Wax + 100% EHA | 0.00792 |
| Centrifuged Wax + 50% EHA | 0.00586 |
| Centrifuged Wax + 25% EHA | 0.00425 |

Similar reduction in Ash content was observed with other acid fractions.

1. A process for the removal of particles from a hydrocarbon stream, the process including the steps of:
1) pre-treating the hydrocarbon stream with an aqueous solution and forming a mixture comprising the hydrocarbon stream and aqueous solution; and
2) introducing the mixture to a centrifuge and separating by centrifugation, from the mixture, a hydrocarbon stream, an aqueous solution and particles.

2. The process as claimed in claim 1, which is a continuous and/or batch process.

3. The process as claimed in claim 1 wherein the particles are metal-containing contaminants.

4. The process as claimed in claim 3, wherein the particles are catalyst fines.

5. The process as claimed in claim 4, wherein the hydrocarbon stream is a wax derived from a Fischer Tropsch reaction.

6. The process as claimed in claim 1, wherein the hydrocarbon stream is pre-treated with an aqueous solution to form a mixture comprising the hydrocarbon stream 5-25% v/v aqueous solution.

7. The process as claimed in claim 6, wherein the hydrocarbon stream is pre-treated with an aqueous solution to form a mixture comprising the hydrocarbon stream 8-12% v/v aqueous solution.

8. The process as claimed in claim 1, wherein the aqueous solution includes an acid and/or reducing agent.

9. The process as claimed in claim 8, wherein the acid is an inorganic acid.

10. The process as claimed in claim 9, wherein the inorganic acid is phosphoric, hydrochloric, sulphuric, acetic, benzene sulphonic, or chloroacetic acid.

11. The process as claimed in claim 8, wherein the acid is an organic acid.

12. The process as claimed in claim 11, wherein the organic acid is a carboxylic acid with the following formula: 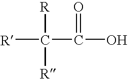
where R, R′ and R″ are each selected from H, or an aryl or alkyl group (linear, branched or cyclic) with a carbon-length of up to C₂₀.

13. The process as claimed in claim 11, wherein the organic acid is formic acid, acetic acid, propionic acid, butyric acid, oxalic acid, 2-ethyl hexanoic acid or citric acid.

14. The process as claimed in claim 13, wherein the organic acid is citric acid.

15. The process as claimed in claim 13, wherein the organic acid is 2-ethyl hexanoic acid.

16. The process as claimed in claim 8, wherein the concentration of the organic acid in the aqueous solution is from 0.05-99.95%.

17. The process as claimed in claim 16, wherein the concentration of the organic acid in the aqueous solution is from 0.5-50%.

18. The process as claimed claims 17, wherein the concentration of the organic acid in the aqueous solution is from 2.5-25%.

20. The process as claimed in claim 1, wherein the centrifuge is operated at atmospheric pressure at a temperature of 60° C. to less than 100° C.

21. The process as claimed in claim 20, wherein the centrifuge is operated at atmospheric pressure at a temperature of about 98° C.

22. The process as claimed in claim 1, wherein the aqueous solution has a density which is within 250 kg/m³ of the density of the hydrocarbon stream.

23. The process as claimed in claim 22, wherein the aqueous solution has a density of 650-850 kg/m³, at 98° C.

24. The process as claimed in claim 23, wherein the aqueous solution has a density of 700-800 kg/m³, at 98° C.

25. The process as claimed in claim 1, wherein the centrifuge is a continuous disc stack centrifuge.

26. The process as claimed in claim 1, wherein the aqueous solution from step 2) is recycled to step 1).

27. A process for the removal of particles from a hydrocarbon stream, the process including the steps of:
1) adding an acid to the hydrocarbon stream to form a mixture of hydrocarbon stream and acid;
2) introducing the mixture containing the hydrocarbon stream and the acid to a centrifuge and separating, from the mixture, a hydrocarbon stream, possibly an aqueous solution, and particles.

28. The process as claimed in claim 27, which is a continuous and/or batch process.

29. The process as claimed in claim 27, wherein the particles are metal-containing contaminants.

30. The process as claimed in claim 29, wherein the particles are catalyst fines.

31. The process as claimed in claim 30, wherein the hydrocarbon stream is a wax derived from a Fischer Tropsch reaction.

32. The process as claimed in claim 27, wherein the hydrocarbon stream separated from the mixture is washed with water.

33. The process as claimed in claim 27, wherein the acid is an inorganic acid.

34. The process as claimed in claim 33, wherein the inorganic acid is phosphoric, hydrochloric, sulphuric, acetic, benzene sulphonic, or chloroacetic acid.

35. The process as claimed in claim 27, wherein the acid is an organic acid.

36. The process as claimed in claim 35, wherein the organic acid is a carboxylic acid with the following formula: 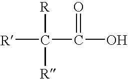
where R, R′ and R″ are each selected from H, or an aryl or alkyl group (linear, branched or cyclic) with a carbon-length of up to C₂₀.

37. The process as claimed in claim 35, wherein the organic acid is formic acid, acetic acid, propionic acid, butyric acid, oxalic acid, 2-ethyl hexanoic acid or citric acid.

38. The process as claimed in claim 35, wherein the organic acid is citric acid.

39. The process as claimed in claim 35, wherein the organic acid is 2-ethyl hexanoic acid.

40. The process as claimed in claim 27, wherein the organic acid added to the hydrocarbon stream has a concentration/purity of from 0.5-100% (w/w).

41. The process as claimed in claim 40, wherein the organic acid added to the hydrocarbon stream has a concentration/purity of from 50-100% (w/w).

42. The process as claimed in claim 41, wherein the organic acid added to the hydrocarbon stream has a concentration/purity of from 90-100% (w/w).

43. The process as claimed in claim 42, wherein the organic acid added to the hydrocarbon stream has a purity of 99.95% (w/w).

44. The process as claimed in claim 27, wherein the centrifuge is operated at atmospheric pressure at a temperature of 60° C. to less than 100×.

45. The process as claimed in claim 44, wherein the centrifuge is operated at atmospheric pressure at a temperature of about 98° C.

46. The process as claimed in claim 27, wherein the centrifuge is a continuous disc stack centrifuge.
